(19) United States
(12) Patent Application Publication
(10) Pub. No.: US 2013/0123862 A1
(43) Pub. Date: May 16, 2013

(54) ARTHRODESIS IMPLANT AND BUTTRESSING APPARATUS AND METHOD

(71) Applicants: Gregory Anderson, Salt Lake City, UT (US); Louis Monaco, Salt Lake City, UT (US); Brock Johnson, Draper, UT (US)

(72) Inventors: Gregory Anderson, Salt Lake City, UT (US); Louis Monaco, Salt Lake City, UT (US); Brock Johnson, Draper, UT (US)

(21) Appl. No.: 13/733,321

(22) Filed: Jan. 3, 2013

Related U.S. Application Data
(63) Continuation-in-part of application No. 13/414,382, filed on Mar. 7, 2012.
Application No. 13/414,382 is a continuation-in-part of application No. 12/901,552, filed on Oct. 10, 2010.

(51) Int. Cl. A61B 17/88; A61B 17/86

(52) U.S. Cl. CPC A61B 17/88; A61B 17/8645
USPC 606/321

(57) ABSTRACT

An arthrodesis anchor as a monolithic piece has prongs supporting barbs extending radially therefrom. A neck between the ends may be angled to accommodate final alignment of resected surfaces of joints to be bonded. The neck may receive a tool for buttressing a first inserted end against further penetration into a joint while the second inserted end is inserted into the other adjacent joint. The proximal and intermediate phalangeal joints may be trimmed and pilot drilled, and may be broached with or without a K-wire guide. A first end may be inserted by a tool, typically into the proximal joint, but the order may be reversed. The second end may then be inserted into a pilot in the base of the intermediate joint.

### RELATED CASES

This patent application is a continuation in part of co-pending U.S. patent application Ser. No. 13/414,382 filed on Mar. 7, 2012 and entitled ARTHRODESIS IMPLANT AND BUTTRESSING APPARATUS AND METHOD, which is a continuation in part of U.S. patent application Ser. No. 12/901,552, filed on Oct. 10, 2010 and entitled ARTHRODESIS IMPLANT APPARATUS AND METHOD, both of which are hereby incorporated herein by reference.

### BACKGROUND

1. The Field of the Invention

This invention relates to orthopedic surgery and more particularly to methods and apparatus for arthrodesis, resecting and fixing bone elements to cause a union for healing in permanent joinder.

2. The Background Art

Orthopedic surgeries originated millennia ago. Anthropological excavations have demonstrated orthopedics practices evidenced in healed, sometimes structurally reinforced, bone healing. Modern orthopedic surgery has greatly advanced the art of improving deformities through surgery. Moreover, the importance of maintaining an active life has motivated demonstrably improved methods for promoting the ability to heal.

For example, patients with a variety of recovery needs were customarily committed to bed rest in hospitals in decades past. Modern medicine realizes the emotional and physical toll that such inactivity takes on a patient. Modern surgical techniques acknowledge the importance of maintaining a physically active body as a mechanism to aid in healing processes. Accordingly, it is desirable to have patients continue in their daily movement and activities as soon as possible. To this end, less invasive surgical techniques have been developed.

Likewise, structural implants such as plates, screws, staples, rods, pins and the like have been augmented by new joint systems, and other orthopedic implants to replace or enhance natural orthopedic structures in the body.

Nevertheless, surgical techniques need to be simplified in many instances. Likewise, the speed at which surgical procedures can proceed is limited by both the physical circumstances of the injury or malady being corrected, as well as the equipment used, and any other supporting equipment or devices required by the procedure.

Accordingly, it would be an advance in the art to provide implants, susceptible to faster installation, more secure holding, post-installation tensioning, and the like. Such features would provide to a surgeon the additional benefit of being able to confirm securement. Implants that can originate and enforce angles of relative positioning between adjacent bone elements are also needed.

It would also be an advance if the anchor could support three dimensional stability following surgery in order to hasten healing, permit early use promoting circulation and healing, and otherwise provide securement with less threat of separation, twisting, disorientation, and the like during the important early days of the healing process.

It would be a further advance of the art to provide three dimensional stabilization between two elements being joined in an orthopedic, and particularly a hammertoe remediation, by providing reliable anchoring in the longitudinal direction, as well as orientation in the lateral and transverse directions orthogonal thereto in order that healing begin early and be promoted by stability of the joint in all three dimensions.

### BRIEF SUMMARY OF THE INVENTION

In view of the foregoing, in accordance with the invention as embodied and broadly described herein, a method and apparatus are disclosed in one embodiment of the present invention as including apparatus and methods for using implants in arthrodesis surgeries. This application incorporates herein by reference U.S. patent application Ser. No. 12/901,552, filed on Oct. 10, 2010, entitled ARTHRODESIS IMPLANT APPARATUS AND METHOD, and U.S. patent application Ser. No. 13/414,382, filed on Mar. 7, 2012, entitled ARTHRODESIS IMPLANT AND BUTTRESSING APPARATUS AND METHOD.

In one embodiment of an apparatus and method in accordance with the invention, an orthopedic anchor, particularly one adapted to arthrodesis, may include two ends of a single, solid member. Typical bone segments may include the phalanges, or proximal and intermediate phalangeal joints, of a patient, such as a patient having a hammertoe deformity.

At each end of an anchor may be axially collinear distributions of barbs, threads, both, one of each, or both sets as barbs. Each set may be oriented along a plane and sized in pitch and depth to promote gripping against not only the structural portion of the marrow or medullar portion of the bone segment into which penetrating, but also to engage the cortical portion of the bone in order to provide a secure longitudinal (axial) loading, urging union of the two joints secured to one another.

In one embodiment, an anchor may be divided at some intermediate neck, between threads distributed proximate one end, and barbs proximate the other. The anchor may be cannulated if straight, and thus rely on K-wire for guidance and stabilization.

Viewed axially an array of barbs, as flutes provides a substantially rectangular cross section. Although circular at their maximum circumference, they have flats formed on diametrically opposed sides. Thus, the fluted, arcuate portions of the barbs may extend laterally in one dimension (two radially opposite directions), but be absent at ninety degrees therefrom.

A slot may be formed in each end of the anchor to extend axially from the tip to proximate the neck, and radially between the two flat aspects of the barbed ends. This geometry forms prongs, each with barbs arrayed axially along its length. In this way, a tool may be fitted against the flats, having a web connecting through the slot between the flats. Various other webs may be formed in tools or barbs in order to stabilize the two prongs or legs of a barbed end with respect to one another, minimize the unsupported length thereof, and the like.

In certain embodiments, the barbs may be non-collinear, with the anchor having a suitable bend built in, such as five, ten, or fifteen degrees between axes on two ends. Accordingly, the length and diameter may be sized to engage the cortical region of the joint penetrated, and the placement may be selected in order to optimize this engagement.

The threads may have a self-tapping, cutting edge. In another embodiment, barbs distributed proximate one end may themselves be angled as if they were threads, but the flat sides render them a form of interrupted threads. This type of barb set may be inserted into a pilot hole in linear axial translation to secure a joint. Alternatively, the set may be threaded into the pilot hole, after which the barb set at the other end may be inserted in linear translation.

Regardless of how inserted, by rotation and linear urging as threads, or simple non-rotated, axial translation as barbs, the set of barbs may be removed by rotating in reverse of the threading-in direction. Thus, typically, one set of barbs (one end, extending radially from a set of prongs) would be interrupted threads, and the other set (opposite end, extending radially from other set of prongs) would have no such angling along its circumference.

When both ends are formed with sets of barbs with angled flutes as interrupted threads, one must be inserted linearly, and both may be, but only one may be threaded in or out, and only while the opposite end set is free and clear of its joint. Withdrawal is not common, and certainly not desirable, but this interrupted-thread embodiment improves a difficult situation, such as failure to join or other problem.

In certain embodiments of an apparatus and method in accordance with the invention, an anchor may be pre-angled to match a tool provided with a web exactly matched to fit any “misalignment” of that angle of the barb with respect to the longitudinal axis of the end first inserted during surgery.

A surgeon may thus maintain surface alignment and contact between the resected contact faces of the bone joints (elements) being joined. Resected at an angle so those faces may be canted slightly with respect to one another, intimate contact will maintain an angle between the longitudinal axes of the two joints, each corresponding to a longitudinal axis of the anchor at its own respective end thereof. This helps maintain the desired orientation of each end of the anchor, in its respective joint, as well as the fit of the contact faces establishing the angle between the axes of the joined joints.

A tool may angle to accommodate the longitudinal axis of a first set of barbs, thus providing axial alignment between the handle of the tool and the longitudinal axis of the set of barbs at the opposite end. Meanwhile, the handle end of the tool aligns at all times with the axis of the end being first inserted, whether having threads, barbs, or interrupted-thread barbs. Likewise, a buttress tool may engage a neck between two ends of an anchor to rotate it, drive it, or resist retreat of a first end while inserting a second end to the anchor.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a perspective view of one embodiment of an apparatus in accordance with the invention including both an anchor and a tool for manipulating the anchor during initial stages of the installation process;

FIG. 2 is a perspective view of the anchor and tool of FIG. 1, viewed from the opposite end thereof;

FIG. 3 is a top plan view of the anchor of FIG. 1;

FIG. 4 is a left side elevation view thereof;

FIG. 5 is an end elevation view from the screw point end;

FIG. 6 is an end elevation view thereof showing the pronged or barbed end that is engaged by the tool;

FIG. 7 is a perspective view of one embodiment of the tool of FIG. 1, having the center portion shortened as indicated in order to provide detail of the handle and anchor ends thereof;

FIG. 8 is a cutaway perspective view of an alternative embodiment of the insertion tool or instrument;

FIG. 9 is a perspective view of a buttress tool used for limiting motion of an anchor during insertion into a second joint;

FIG. 10 is a perspective view of one embodiment of a broaching tool suitable for enlarging a pilot hole in a joint for rendering a joint suitable to receive an apparatus in accordance with the invention;

FIG. 11 is a perspective view of a K-wire rod having both ends surgically sharp for insertion into the medullar portion of a joint for effecting a surgical method in accordance with the invention;

FIG. 12 is a perspective view of one embodiment of a broach traveling along a K-wire in one embodiment of a method in accordance with the invention;

FIG. 13 is a perspective view of an alternative embodiment of an anchor in accordance with the invention relying on two barbed ends, and having a pre-formed bend angle between the longitudinal axes thereof;

FIG. 14 is a perspective view of one embodiment of a cannulated anchor having a monolithic and homogeneous material and having barbs on both ends, in this embodiment all barbs are radially extending in the same direction;

FIG. 15 is an end view of the anchor of FIG. 18;

FIG. 16 is an end view of the anchor of FIG. 20;

FIG. 17 is a perspective view of an alternative embodiment of one embodiment of a double-barbed anchor, homogeneously formed of a single material and having the barb sets at opposite ends thereof extending radially at directions that are circumferentially orthogonal to one another, these being 90 degrees out of phase;

FIG. 18 is a perspective view of an alternative embodiment of an apparatus having a bent neck between the two ends, and thus angling the axes central to the sets of barbs on either end at an angle of a suitable amount for effecting an angular displacement of one joint with respect to another for joinder;

FIG. 19 is a perspective view of an alternative embodiment of an apparatus, cannulated, having threads and pitched barbs;

FIGS. 20A and 20B are front and back end elevation views thereof;

FIG. 21 is a top plan view thereof;

FIG. 22 is a side elevation view thereof;

FIG. 23 is a perspective view of an alternative anchor in accordance with the invention having barbs at both ends;

FIGS. 24A and 24B are front and rear elevation views thereof;

FIG. 25A is a top plan view thereof;

FIG. 25B is a right side elevation view thereof;

FIG. 26 is a perspective view of an anchor in accordance with the invention showing the process of engagement of a proximal end with a first bone joint;

FIG. 27 is a perspective view thereof, showing the buttressing tool in place to resist any further retreat into the first joint;

FIG. 28 is a perspective view of an anchor, having a first end inserted, as a second end is being inserted into a second bone joint (that is to be fused with the first bone joint) while the buttressing tool stabilizes the anchor in the first joint;

FIG. 29 is a perspective view of the phalanges in an alternative embodiment of a method for joinder, relying on a K-wire inserted in the pilot to act as a guide and structural member;

FIG. 30 is a perspective view of the K-wire in place, and a drilling tool being guided by the K-wire to form a larger opening for receiving an anchor;

FIG. 31 is a perspective view of a tool prepared to insert an anchor therein; and

FIG. 32 is a perspective view of the joints fully reduced.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

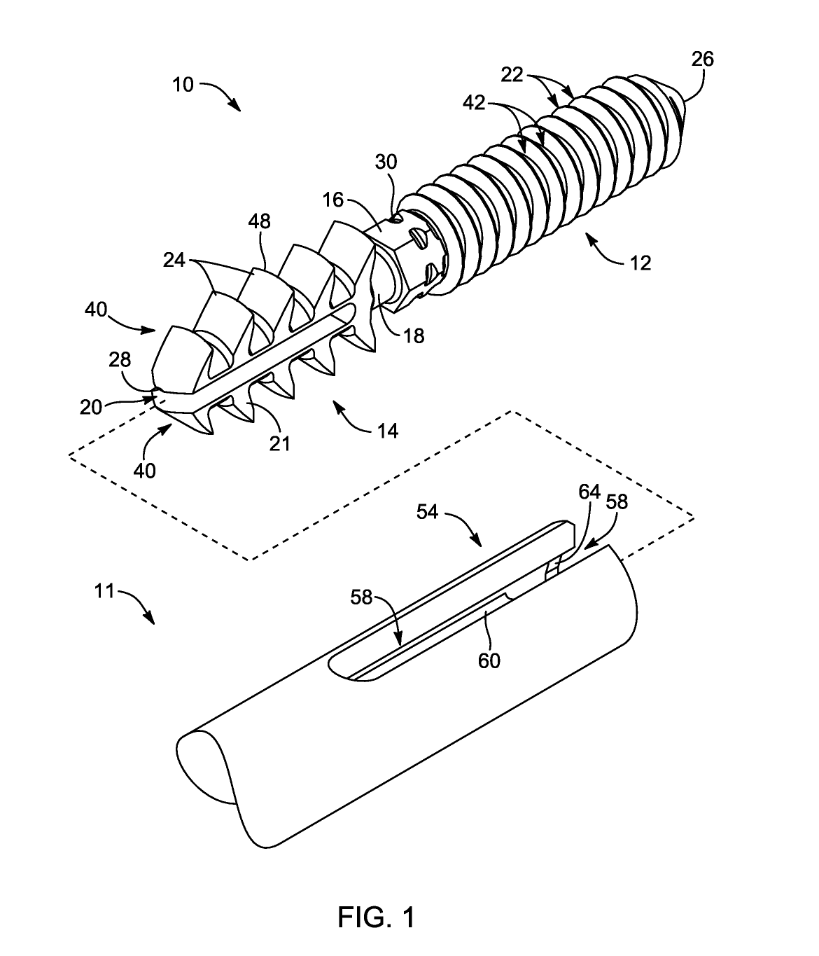

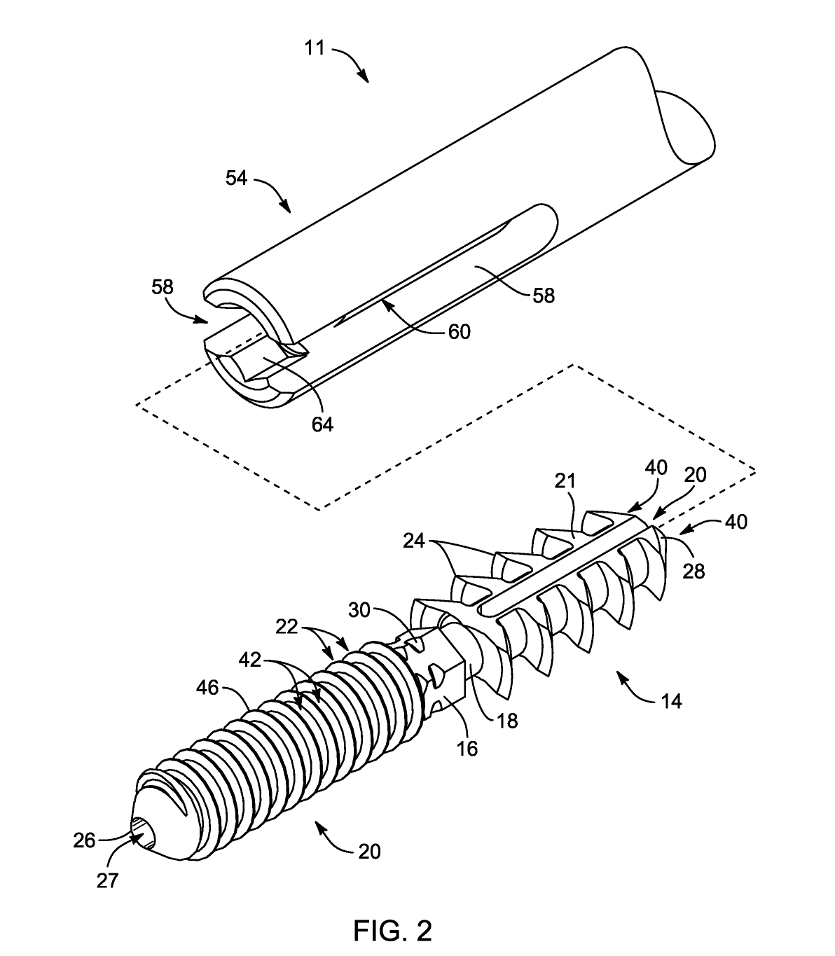

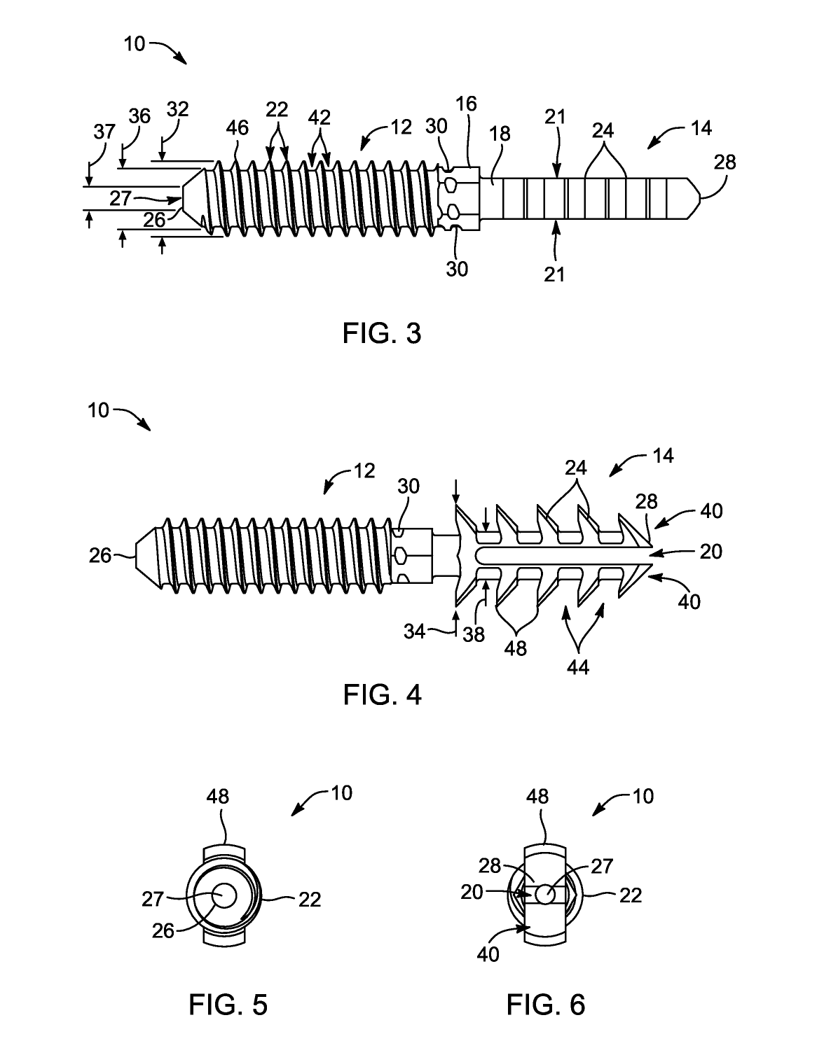

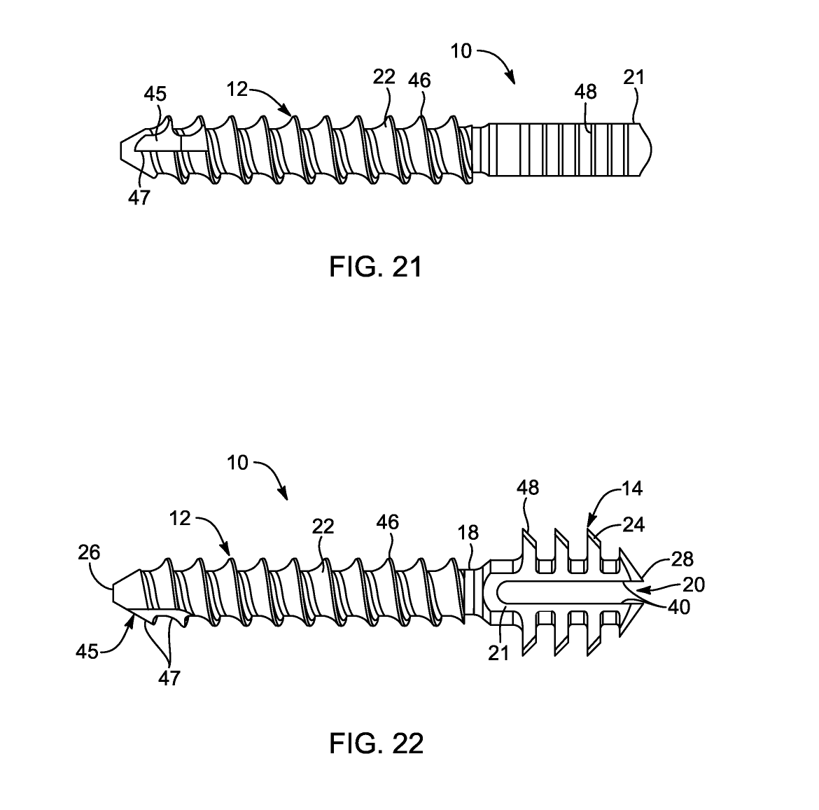

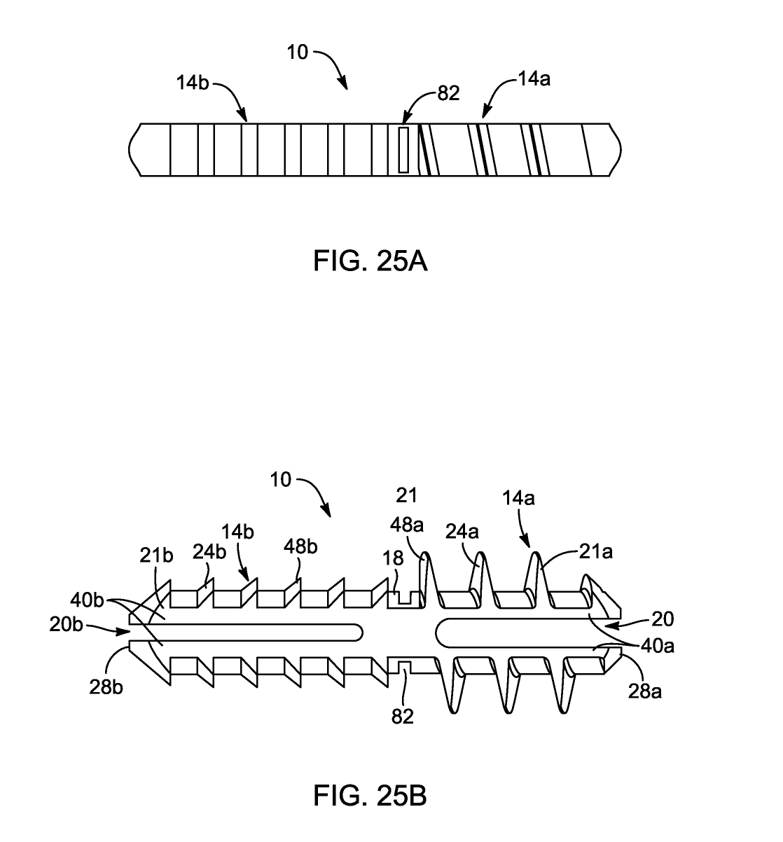

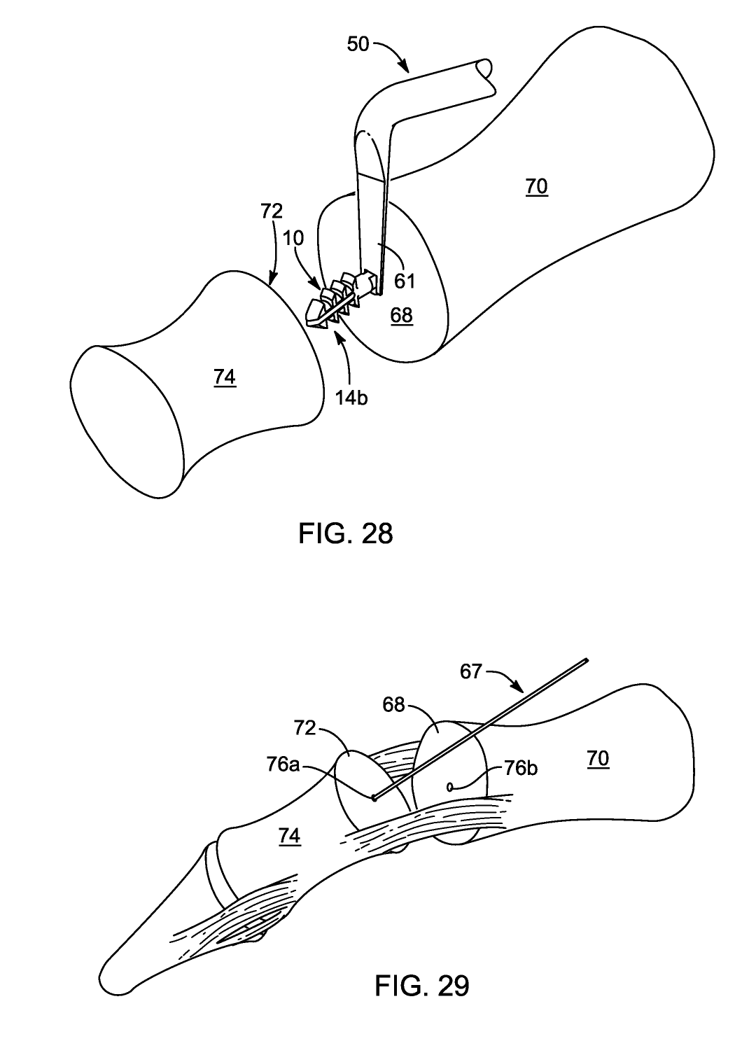

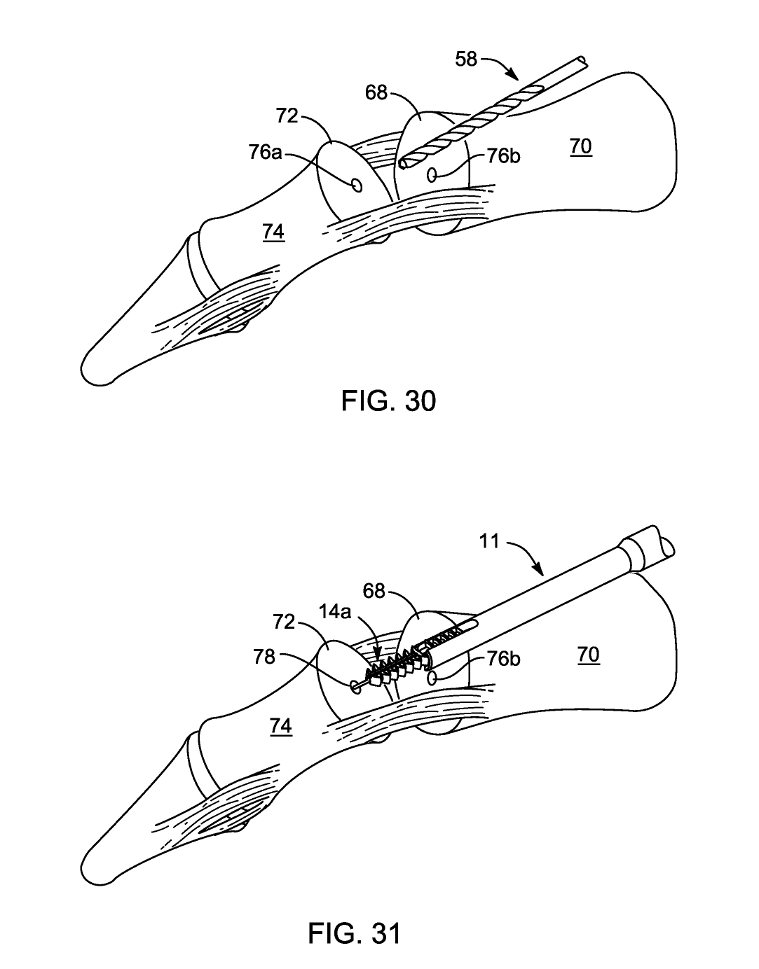

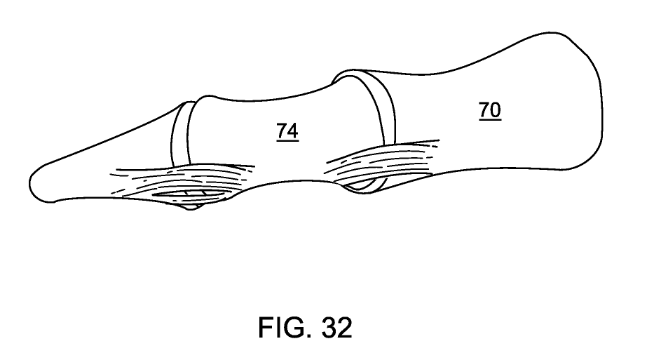

Referring to FIGS. 1 through 6, while referring generally to FIGS. 1 through 32, a system 10 or apparatus 10 in accordance with the invention may include an anchor 10 that may be manipulated and applied using a tool 11. The anchor 10 includes a screw portion 12 or a screw 12 formed in a unit with a barb portion 14 or barb 14. In the illustrated embodiment, the screw 12 and barb 14 portions are formed together of a single, homogeneous material simultaneously formed in any suitable manner.

In certain embodiments, the apparatus 10 may be formed to be of a biodegradable material. Nevertheless, in other embodiments, durable materials that are not rejected by the body may be used. For example, stainless steel, titanium, and the like have been found suitable for applications useful for anchors 10 in accordance with the invention.

The anchor 10 may also be formed to include a shank 16 or a shank portion 16. In certain embodiments, it has been found that the shank 16 is best located immediately adjacent the screw portion 12. In application, the screw 12 may be threaded into a portion of bone, typically the medullar portion. In addition to the shank 16, the anchor 10 may include a shaft 18 or neck 18 between the first inserted portion 12, which may be a screw portion 12 and the later inserted portion 14 or barb portion 14.

In some embodiments, the shaft 18 may be pre-angled in order to provide a known and desired angular difference between the orientation of the portion 12 and the portion 14 of the anchor 10 once installed. In this way, a pre-determined angle may exist in the shaft 18.

In the illustrated embodiment, an anchor 10 may include a slot 20. The effect of the slot 20 is to form a cantilevered, pronged arrangement. Also, the slot 20 may serve to engage the tool 11 in order to drive the anchor 10 into a bone joint for a surgery. The slot 20 may also provide a region in which tissue will reform and fill up, further stabilizing the anchor 10 once in place. Typically, an anchor 10 is not removed. Rather, the anchor 10 stabilizes a surgery, and remains in place after healing is complete.

A screw portion 12 may include threads 22 at a selected pitch, depth, advance angle, and the like, which threads 22 may be buttressed such that they tend to be supported more against force or deflection in one direction than another.

By the same token, the barb portion 14 may include a plurality of barbs 24, the barbs may act on cantilevered arms in order to pass through a pilot hole, and then work out into the medullar region of the bone, ultimately engaging the cortical portion thereof. Likewise, barbs 24 are best engaged into cortical material of the bone, the outer shell like portion that is more dense, stronger, and much harder than the medullar or central region of the marrow.

In order to facilitate an insertion, the screw end 12 may have a point 26. Also, the screw 12 may be cannulated or hollow. Thus it may not form a sharp point 26.

Likewise, the barb portion 14 may include a point 28 associated with the first of the barbs 24. Like the opposite end 12 of the anchor 10, the point 28 at the barb portion 14 of the anchor 10 may be sized to fit or otherwise engage a pilot hole. A pilot hole could be simply drilled at a single diameter or broached larger, and the barb point 28 may engage that resulting hole. An anchor 10 in accordance with the invention may be radial cavities 30, be cannulated 27 along the entire length, or both.

A screw 12 may have an outer diameter 32 defining the outermost edge of the flutes or threads 22 thereof. Meanwhile, the outermost diameter 34 of the barbed portion 14 will typically follow an arcuate path, although not usually for a full circle. That is, the barbs 24 are formed to present a flat aspect 21 that assists in stabilizing the barbs in place, reduces the requirement in the size of the pilot hole required, and also provides for cantilevering of the barbs 24 in order that they may ultimately extend to their maximum outside diameter 34 to engage the cortical portion of the bone.

Meanwhile, the threads 22 have an inner diameter 36 that defines the valley, trough, or the relief that exists at a lesser diameter than the outer diameter 32 of each of the flutes 22 or threads 22. Moreover, the screw 12 may also have an innermost diameter 37 that represents a cavity or cannulation 30 formed as a tubular vacancy along the center of the anchor 10.

As with the screw portion 12, the barbed portion 14 has an outer diameter 24 as well as an inner or minor diameter 38 with the cannulation 30 forming an inside diameter 37. The inner diameter 38 provides for additional material, provides for a smooth and arcuate surface, and provides additional stiffness for the barb portion 14. Effectively, the barb portion 14 is divided into two prongs 40. Each of the prongs 40 contains an array of barbs 24, the first one representing the point 28.

Thereafter, the barbs 24 may increase in diameter or simply have a larger outer diameter 34 than the first barb. Each of the barbs 24 has an outer diameter 34 at which it engages the bone, and an inner diameter 38 that represents effectively the outermost fiber of a beam that cantilevers or carries the barb 24. That beam 40 is one of the prongs 40 arrayed with barbs 24 therealong.

Thus, each of the threads has a gap 42 or pitch 42 between threads. Similarly, each of the barbs 24 has a pitch 44 or maximum gap 44 as a center-to-center or edge-to-edge distance 44.

In contrast, the gaps 44 may initially be less filled with bone material inasmuch as the barbs 24 must pass therethrough. However, the cantilevered effect of the prongs 40 permits the barbs 24 to move toward one another, forced by the pressure of the surrounding bone. Nevertheless, upon the slightest provocation to retreat, or upon coming to rest, the prongs 40 are urged apart by their inherent elasticity, causing each of the barbs 24 to move out toward its edge 48. The edges 46 of the screws 12 or the edges 46 of the threads 22 will cut into and anchor against the bone material, and particularly against cortical material. Likewise, the edges 48 of the barbs 24 will tend to advance outward as they come to rest, cutting through the medullar material and engaging the cortical material of the bone.

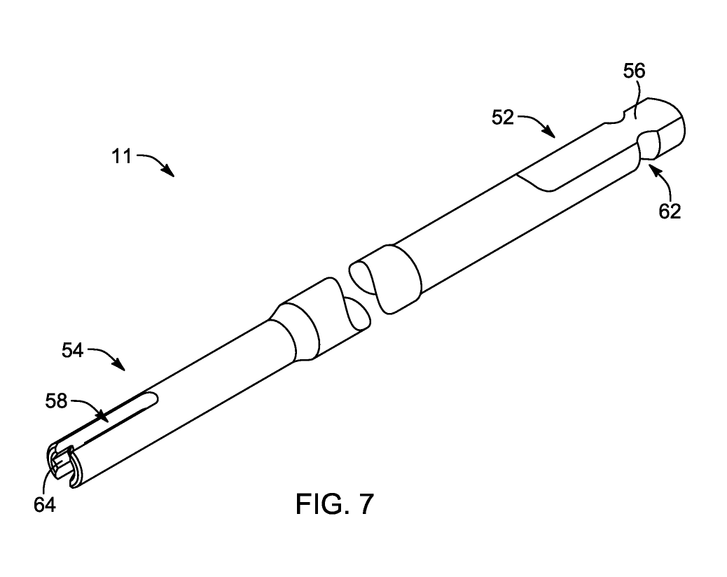

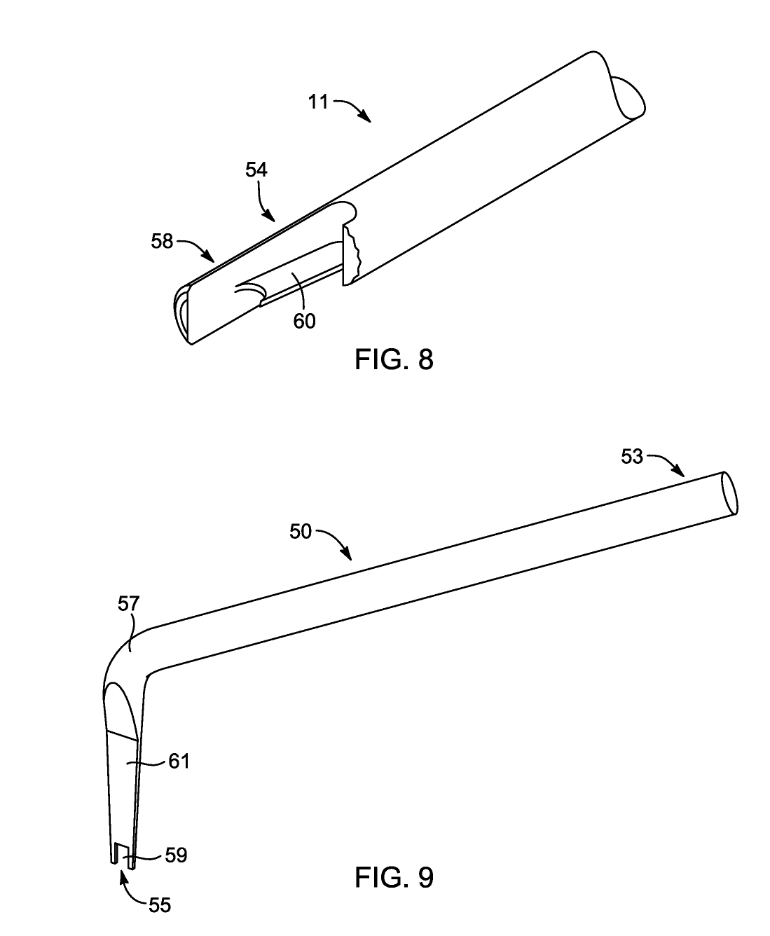

Referring to FIGS. 7 and 8, while continuing to refer generally to FIGS. 1 through 32, a tool 11 in accordance with the invention may be formed with a receiving portion 54 to fit over part of the anchor 10. Accordingly, opposite the receiving portion 54 of the tool 11, a handle portion 52 may be adapted to be gripped by a hand of a surgeon. For example, the handle portion 52 may include a flat 56 as well as relief 62 for gripping.

Any time relief is provided, such as by notching, knurling, or the like, the handle portion 52 may be more readily gripped because flesh from the hand of a user expresses into the slot or relief area providing better than a frictional grip thereon. Accordingly, the tool 11 may include a flat 56, a relief slot 62, knurling, or the like in order to provide better grip of the tool 11 for a surgeon. Accordingly, a surgeon user may thereby handle with greater security the tool 11 in order to drive the anchor 10 into a bone joint 70 where the anchor 10 will serve to stabilize the conjoining of two joints 70, 74 during healing.

Likewise, the anchor portion 54 or the receiving portion 54 of the tool 11 may include a slot 58 to receive the back or later inserted barb portion 14 of the anchor 10. Specifically, the prongs 40 that form the main beams 40 of the rear or later inserted barb portion 14 may fit within the slot 58, sized to receive it.

In certain embodiments, a web 60 may extend between opposite faces of the slot 58 in order to engage the slot 20 in the anchor 10. Thus, the barb portion 14 is engaged on the flats 21 by the opposing faces that form the slot 58. Meanwhile, the slot 20, and the inner faces of the secondary, back, or later inserted barb portion 14 that form the slot 20, are engaged by the web 60. The web 60 may be angled so the first-inserted portion 12 is aligned, or even coaxial or collinear, therewith.

In certain embodiments, a relief 64 may be formed to receive a hexagonal or other shape of the shank 16. Thus, the anchor 10 may be engaged by the tool 11 by the slot 58 engaging the flats 21 of the barb portion 14, the web 60 engaging the faces of the slot 20 in the barb portion 14, and the relief section 64 fitted to the shank 16 for engaging the shank 16 therein.

In an apparatus and method in accordance with the invention, a tool 11 or instrument 11 may be formed to serve as an anchor 10 that has a pre-determined offset angle between the longitudinal axes of the barb portions 14a, 14b of the anchor 10. In one embodiment of an apparatus and method in accordance with the invention, the tool 11 may be formed to receive an anchor 10 having a pre-determined angle of bending of the shaft 18 or neck 18 thereof.

Thus, the web 60 is oriented at the same angle as that in the anchor portion 54 of the tool 11. The angle provides the direction required to accommodate a second inserted barb portion 14b of an anchor 10. Placing that end 14b of an anchor 10 into the anchor portion 54, and the slot 58 particularly, of the tool 11, the first inserted portion 14a is aligned parallel with the handle 52. The longitudinal axis thereof is in alignment, and preferably collinear with, the longitudinal axis of the handle portion 52 of the tool 11.

Upon completing the process of inserting a first inserted portion 14a of the anchor 10, a surgeon may draw the handle portion 52 away from the anchor 10, and thus remove the slot 58 and web 60 from engagement with the second inserted barb portion 14b of the anchor 10.

Line-of-sight alignment may be enhanced by the handle progressing continuously in the longitudinal direction. Thus, with the first inserted portion 14a extending therefrom, natural line of sight and natural eye-to-hand coordination may aid orientation of the tool 11.

Extraction of the tool 11 after installation of the anchor is along the longitudinal axis of the second inserted barb portion 14b. For example, the slot 58 of the tool disengages from the flats 21 of the barbs 24, while the slot 20 of the barb portion 14b disengages from the web 60 of the tool 11.

The tool 11 is configured to receive one end 14a, 14b of the anchor 10, fitted in the slot 58, with the slot 20 of the anchor fitted over the web 60. The web 60 is angled to match the pre-set angle in the neck 18 of the anchor 10, such that the remaining end 14b, 14a is oriented collinearly with the tool 11 for insertion.

That pre-set angle between the two barb portions 14a, 14b is best formed in the neck 18 of anchor 10 at manufacture and not achieved by bending the neck 18 at anytime thereafter. This is usually so, regardless of which types of ends 12, 14 are used. It is even more important if the anchor 10 is cannulated. Typical pre-set angles of zero, five, ten and fifteen degrees may be used to accommodate the relative angle needed between two joints 70, 74. Most commonly, ten and 15 degree angles are used if any angle at all. Accordingly, the apparatus and method of each embodiment may be employed in a manner similar to that for the other embodiments, with variations only as required.

Referring to FIG. 9, a buttress tool 50 may be formed with a handle 53 or handle portion 53 extending at sufficient length to be easily grasped by a hand of a surgeon. A corner 57 or angle 57 between the handle 53 and the main face 51 provides visibility of the face 61 while holding the handle 53 at a convenient angle in use.

At the distal end 55 or extremity 55 of the face 61 away from the handle 53, a slot 59 is formed in the face 61. The size and shape of the slot 59 are formed to engage a receiving portion of the neck 18 or shaft 18 on the anchor 10.

The anchor receiving portion 55 or the operational end 55 of the tool 50 is inserted into the space between two joints during surgery. Accordingly, the slot 59 engages the neck 18 by way of a slot 82 or notch 82. The tool 50 in the slot 82 is located at a position effective to hold the anchor 10 from proceeding any further into a first joint (e.g., 70, 74).

For example, when a second joint (e.g., 74, 70) is forced onto the opposite end 14a, 14b of the anchor 10, the face 61 against the first joint (e.g., 70, 74) precludes any motion of the anchor 10 further into that first joint (e.g., 70, 74). Accordingly, the second joint may then be engaged onto the second end 14b, 14a of the anchor 10 until the second joint comes flush against the face 61. At that point, the mere thickness of the anchor-receiving end 55 of the tool 50 remains between the joints. That end 55 is comparatively thin, and may actually be less than the pitch between the barbs 24.

Thus removing the buttress tool 50 makes possible the movement of either end 14a, 14b of the anchor 10 into its respective joint. However, any favoring of one over the other is not problematic, because the overall distance (e.g., thickness of the end 55) remaining is insignificant.

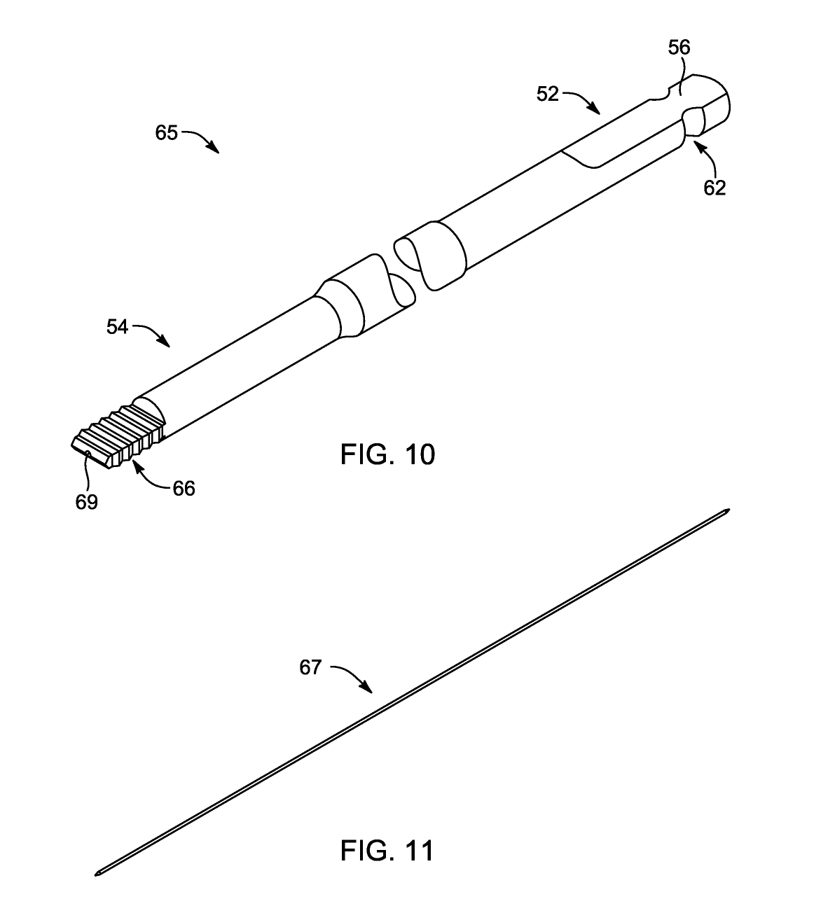

Referring to FIG. 10, a broach 65 or a broaching tool 65 may include a handle portion 52 and a forward end 54 holding the actual broaching region 66 or cutters 66 for broaching an aperture in a joint. Thus one may properly speak of the broach 65 or the broach 66.

In the illustrated embodiment, the broach 66 may be of any suitable size, and multiple tools 65 may be used. One may rely on a sequence of broaches 66 of progressively larger sizes. Thus, initially, a surgeon may begin with a pilot or pilot hole 76, formed with an awl or a drill. Subsequently, various sizes of broaches 66 may be inserted and drawn axially back and forth repeatedly. Each further opens the aperture 78 initially formed as a pilot 76. Ultimately, the broached opening 78 or canal 78 is shaped to be the size and shape of the largest broach 66 used, and of a suitable depth and dimension for an anchor 10 selected.

Referring to FIG. 11, a K-wire rod may be used, an includes double trocars (i.e., pointed ends) as well as the main shaft 67 or rod 67 thereof. In some embodiments, the K-wire 67 may be used exclusively during surgery as a penetrating guide. In other embodiments, the K-wire 67 may be left in place after completing an arthrodesis reduction. In such case, it may be withdrawn after the degree of healing has reached a predetermined stage.

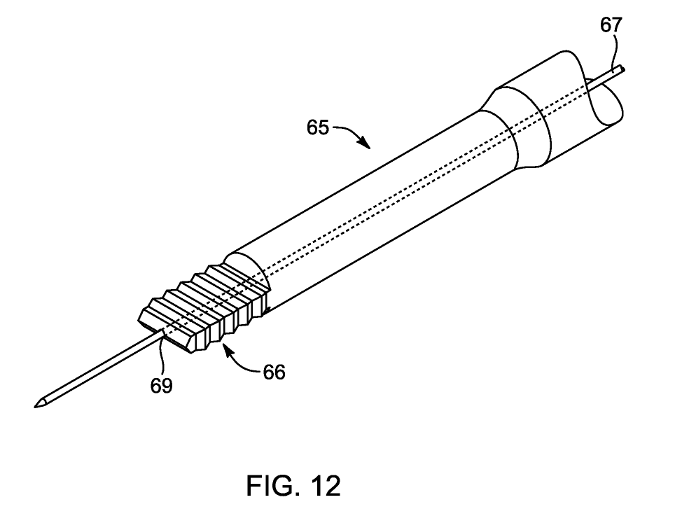

Referring to FIG. 12, the broaching tool 58 may include a cannula 69 sized to freely receive the K-wire 67. In operation, the K-wire 67 may penetrate into a pilot 76, thus acting as a guide 67 along which the cannula 69 will slide. Accordingly, the broach 66 or broach cutters 66 may thus be guided.

This ensures that the broached opening 78 formed thereby is not misaligned or oversized. An oversized opening 78 will not be suitable to receive and engage the barbs 24 of an anchor 10 if oversized too far. The broaching tool 58 may slide forward and backward along the K-wire 67, thus alternatively penetrating into the medullar portion of the joint 70, 74 and then retreating with the cutters 66 drawing out the cuttings from the broached opening 78.

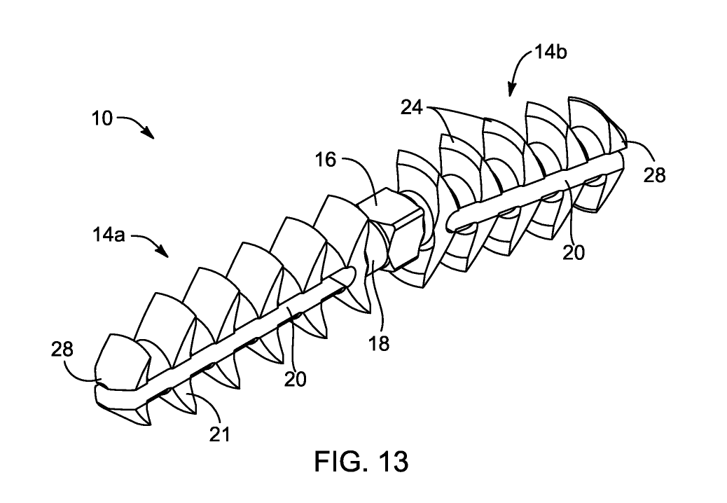

Referring to FIG. 13, in one embodiment of an apparatus 10 in accordance with the invention, the neck 18 of the anchor 10 may be angled in order to offset by a predetermined angle the first end 14a of the anchor 10 with respect to the second end 14b. In the illustrated embodiment, the barbs 24 in the two sets 14a, 14b or distributed along the two ends 14a, 14b are angled with respect to one another. Each defines a central axis, which for each end 14a, 14b will be offset from the other end's axis by some angle, typically five, ten, or fifteen degrees (usually ten or fifteen).

The illustrated embodiment shows the two ends 14a, 14b or the sets 14a, 14b of the barbs 24 being oriented to extend radially in the same directions. Thus, one may think of the two sets 14a, 14b of barbs 24 being coplanar, or to have an axis of symmetry. The plane of a flat on the side of each barb would be coplanar in each set 14a, 14b. Meanwhile, the barbs 24 at each end 14a, 14b are both required to be inserted axially into respective joints.

A function of an anchor 10 in accordance with certain embodiments is to provide a change in the relative angle between the resected joints in an operation. Thus, a surgeon may resect a face or head of a joint as appropriate at a specific angle in order to ensure that, for example, the intermediate joint 74 may be angled downward with respect to a proximal joint 70. In this way, the sets 14a, 14b of barbs 24 can each extend axially in alignment with their respective joints 70, 74 while yet maintaining the resected faces 68, 72 in compression, with the anchor 10 being in tension to maintain the force therebetween.

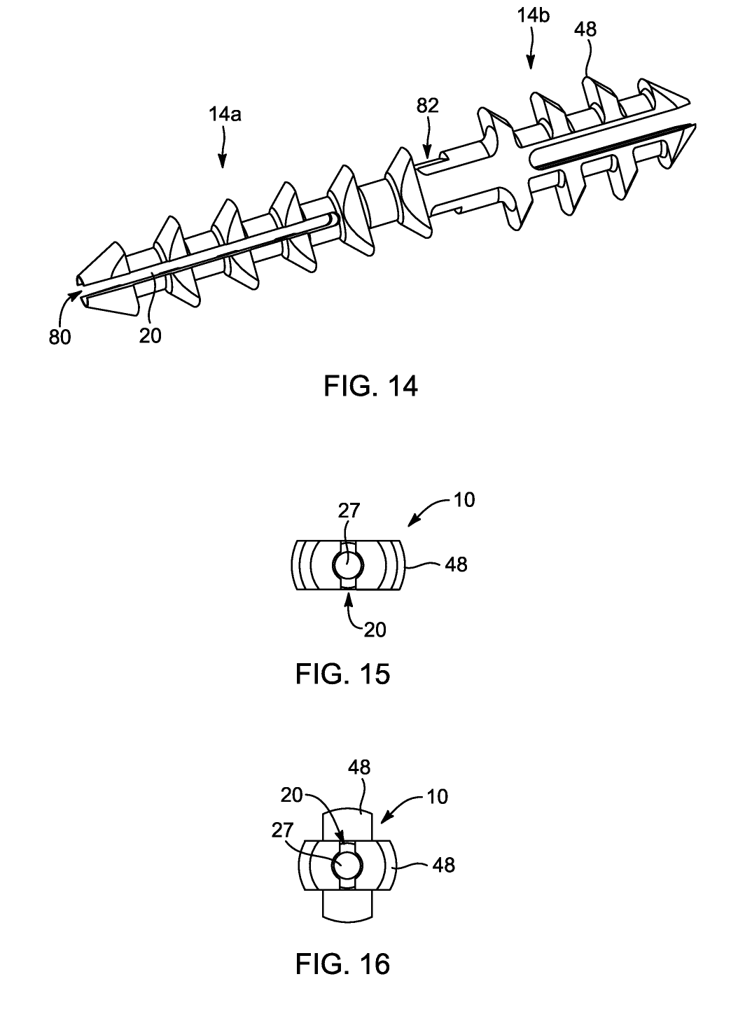

Referring to FIGS. 14 and 15, an alternative embodiment of an anchor 10 in accordance with the invention may be completely straight. In the illustrated embodiment, the barbs 24 have edges 48 oriented in the same direction. In this embodiment illustrated, the anchor 10 is provided with a cannula 27. In this embodiment, the neck 18 is also illustrated with a slot suitable for receiving the slot 58 of the tip 55 or the receiving end 55 of the buttress tool 50.

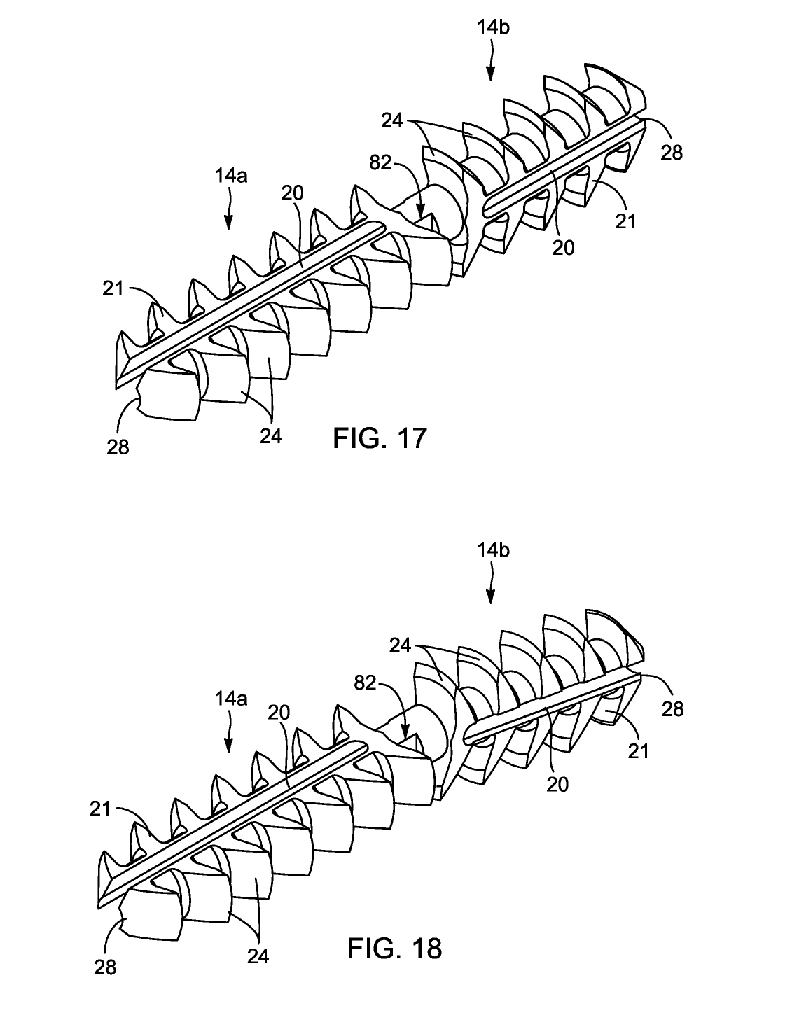

Referring to FIGS. 16 and 17, an anchor may also be cannulated, and yet have the barbs extending in different directions from one another. In this embodiment, the barbs 24 of a first end 14a are rotated circumferentially (about a central axis) by 90 degrees with respect to the barbs 24 on the opposite end 14b. Inasmuch as this embodiment of an anchor 10 is straight, a cannula 27 may be formed and used profitably, such as to receive a K-wire 67 as a guide in the cannula 27 thereof.

In the embodiment of FIGS. 14 and 15, and embodiments in which barbs 24 are all aligned the same at both ends 14a, 14b, the barbs 24 are all extending in the same direction and thus bear on the same cortical aspect of their respective joints 70, 74. In contrast, in the embodiment of FIGS. 16 and 17, the barbs 24 at opposite ends 14a, 14b will engage aspects rotated 90 degrees with respect to one another. For example, one set of barbs will engage a side portion of a joint 70, 74 while the opposite end will engage the top and bottom portions of a cortical portion of a join 70, 74.

Referring to FIG. 18, an alternative embodiment of an anchor 10 in accordance with the invention includes a bend in the neck 18, thus offsetting the central axes of sets 14a, 14b of barbs 24 or opposite ends 14a, 14b from one another at a preselected angle. Such an embodiment does not receive a K-wire 67 as such a rod 67. A K-wire 67 could not slide through a cannula 27. Accordingly, no cannula is provided in this embodiment.

Insertion of the anchor 10 is nevertheless accomplished in the same manner as that of straight configurations. The angle between the opposing ends 14a, 14b is accommodated by the web 60 in the tool 11. That is, the web 60 is angled within its slot 58, thus receiving the anchor 10 therein and the slot 20 in the anchor 10 is accommodated with the angled web 60. Thus, insertion is always in a respective axial direction for the anchor 10 and the tool 11. The tool 11 still drives the first inserted end, whether the end 14a or 14b at a collinear and straight angle with respect to the axial direction of the tool 11.

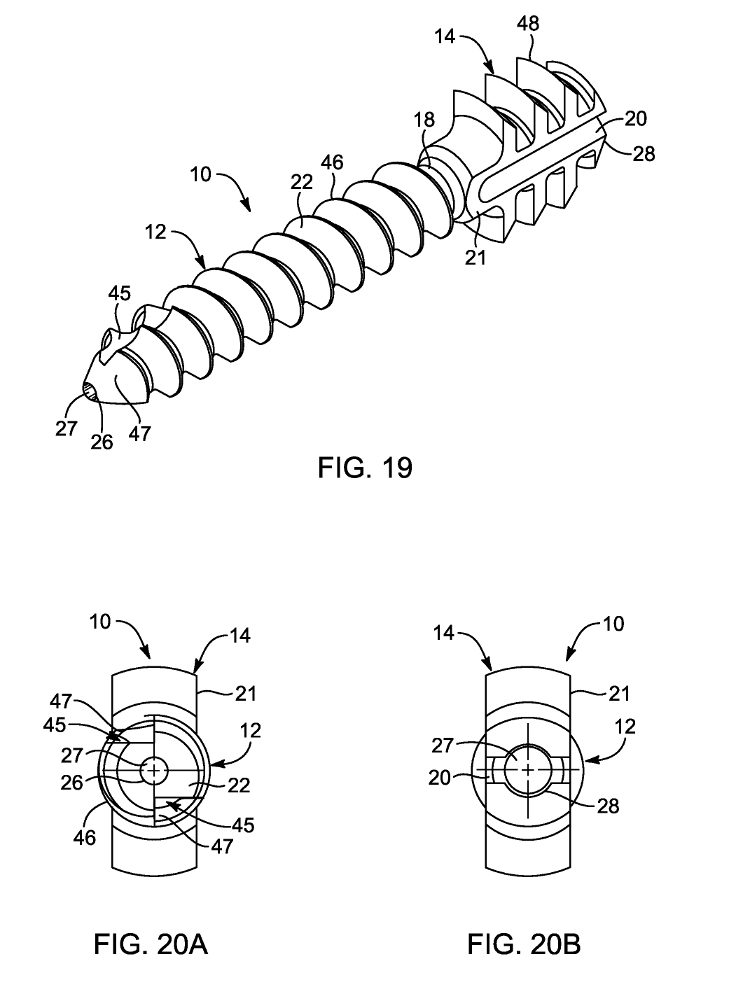

Referring to FIGS. 19 through 22, in one embodiment of an anchor in accordance with the invention, the screw portion 12 may include a relief region 45 or excavation portion 45 from which the material of the threads 22 may be removed. The result is a cutting edge 47 that makes the screw portion 12 self tapping. In the illustrated embodiment, the screw portion 12 of the anchor 10 may be urged into a pilot hole in a joint (bone segment) by axial force and rotation in combination. The axial force along the center line or axis of the anchor 10 tends to keep the flutes 22 or threads 22 engaged.

For example, the edge 46 will tend to engage the bone, initially in the medullar portion, and eventually in a cortical portion. Meanwhile, the cutting edge 47 and excavation region 45 will tend to cut threads or tap a matching thread pattern into the bone, for receiving the thread portion 12. This typically results in less damage to the bone, less compressive force required axially, and reduced loads resisting rotation of the anchor 10 when threaded into a joint.

The anchor 10 also includes a barb portion 14 having barbs 24 whose edges 48 likewise are driven together as the prongs 40 are forced toward one another as the anchor 10 is inserted into a pilot hole 76, whether that pilot hole is broached or not. In this embodiment, the barbs 24 are aligned and distributed axially to be collinear with one another.

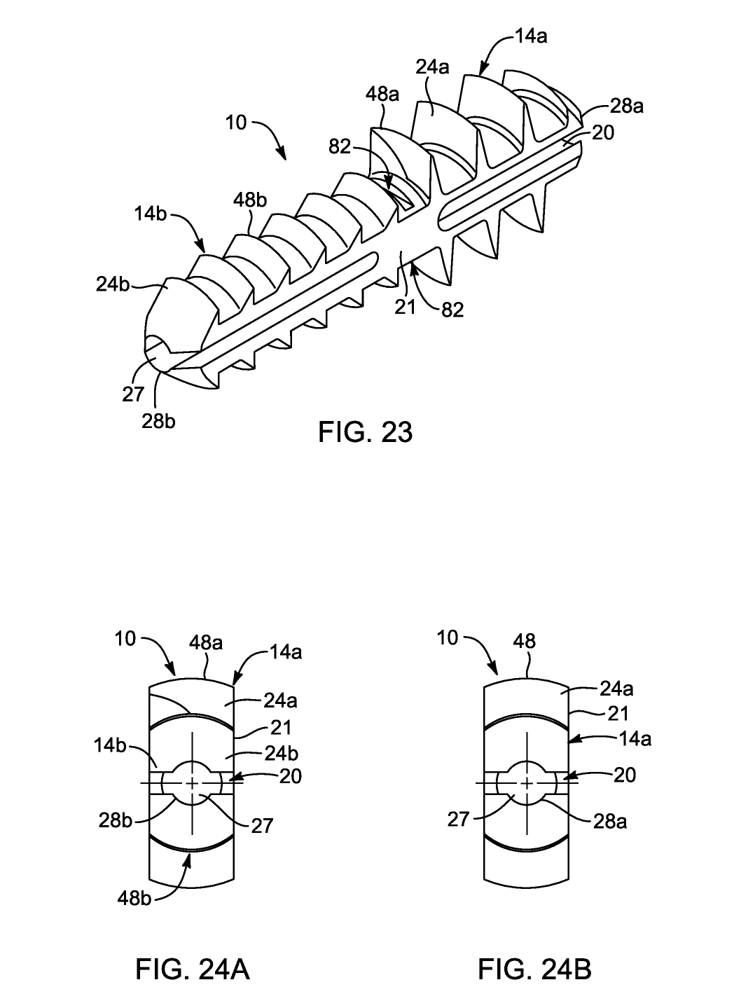

Referring to FIGS. 23 through 25, while continuing to refer generally to FIGS. 1 through 32, in certain embodiments, the barb portion 14a of an anchor 10 having two barbed portions 14a, 14b, may have an angled barb 24a. That is, each of the barbs 24a has an advancing pitch such as a screw thread. However the flats 21 of the barb portion 14a still are provided. Accordingly, the angled or pitched barbs 24a may operate as interrupted threads 24a.

For example, if the joint 70 (bone segment 70) of a patient is determined to be comparatively hard and strong, then a surgeon may thread the barbed portion 14a into a pilot hole in the joint 70. By contrast, if the medullar portion of the joint 70 is comparatively softer, then the surgeon may insert the barbed portion 14a directly by linear translation into a pilot hole, or into a broached opening 80. In any event, in a case where an anchor 10 must be removed, the interrupted thread 24a may provide substantially easier removal, making a threaded passage for its own removal, and urging itself outward upon turning backwards, in the direction opposite to which it would be threaded into a joint 70.

Meanwhile, the barbed portion 14b operates the same as in other embodiments described hereinabove. Having no angle or twist to the barbs 24b, this set 14b or portion 14b passed its barbs 24b into a pilot hole 76, or a broached opening 80 by direct linear translation. In the illustrated embodiment, the flats 21a may have comparatively sharp edges where they interface with the flutes 24a or barb edges 48a of the barbs 24a. Accordingly, whether being threaded inward or outward, the interrupted threads 24a or barbs 24a with their angled configuration will work their way into or out of the joint according to the circumferential direction in which they are turned.

As in other embodiments described hereinabove, the prongs 40 tend to compress toward one another as cantilevers. This response to being forced into bone is partly the result of the faces of the barbs 24a being forced together due to their wedge-like shape. Meanwhile, when being threaded into a joint 70, there will still be a tenancy to draw the prongs 40 together, even though it will be reduced somewhat by the cutting action of the flats 21a advancing through the joint.

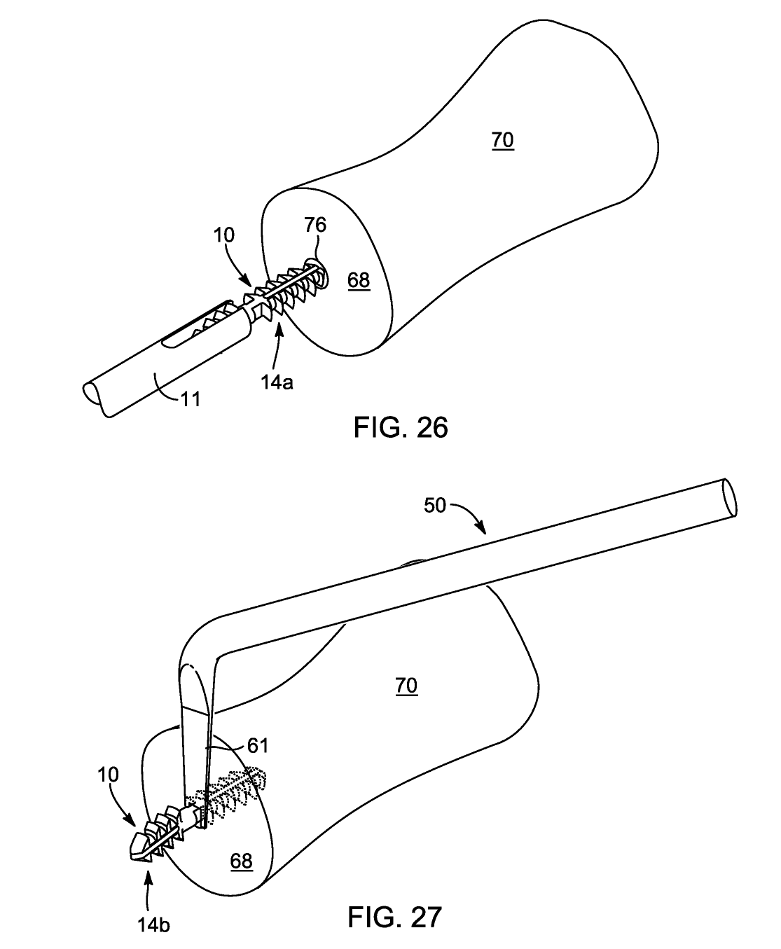

Referring to FIGS. 26 through 32, while continuing to refer to FIGS. 19 through 25, and FIGS. 1 through 32 generally, a surgical procedure may rely on a variety of techniques. For example, in the embodiment of FIGS. 19 through 22, when applied to a joint 70 as in FIGS. 26 through 28, may begin by inserting the anchor 10 into a tool 11 such that the flats 21 and the slot 20 are engaged by the slot 58 of the tool 11, and the web 60 thereof, respectively. In the illustrated embodiment, the thread portion 12 would be inserted first into the joint 70, beginning at the resected face 68. The tool 11 would be rotated in order to thread the thread portion 12 into the joint 70. Upon arriving at the proper depth of penetration, the threads 22 and the threaded portion 12, in fact the entire anchor 10 may cease rotation, and the tool 11 may be removed. Thereafter, the buttress tool 50 may be used. Referring to FIG. 27, the buttress tool 50 may be placed on the neck portion 18 of the anchor 10, thus assuring that the orientation of the barb portion 14 is maintained, and the thread portion 12 is not permitted to penetrate into the joint 70. The barb portion 14 of the anchor 10 of FIG. 19 may then be inserted into a pilot hole 76 or broached opening 80 in a second joint 74 in order to complete the insertion of the anchor 10. The buttress tool 50 may be removed once the second joint 74 contacts the face 61 thereof. Manual completion of the reduction may then proceed. At that point, so little additional distance remains between the faces 68, 72 of the joint 70, 74, respectively, that it is not problematic if the penetration goes either way, that is, into either joint 70, 74, or both.

Referring to FIGS. 26 through 28, with respect to the embodiment of an anchor 10 and in accordance with FIGS. 23 through 25, an anchor 10 in this embodiment may be received by the barb portion 14b being placed into the tool 11. Thus, if the medullar portion of the joint 70 (see FIGS. 26 through 32) is comparatively softer, than the barbed portion 14a may simply be forced by linear axial motion into the joint 70, by means of a pilot hole 76 drilled aperture 78 or broached opening 80. Typically, only a large pilot canal 78 is required. The prongs 40a tend to draw together in response to the wedge like action of the barbs 24a, if the anchor 10 is advanced linearly without any rotation.

In an alternative embodiment of a procedure, the tool 11 may apply axial force along the central axis of the anchor 10, while the tool 11 also engages the slot 20b and flats 21b of the barbed portion 14b. This loading provides both linear force in an axial direction to urge translation, along with rotation of the anchor 10. It encourages a thread-like advance by the angled barbs 24a of the barbed portion 14a. Thus, in situations where the bone is comparatively harder or stronger, the angled-barb portion 14a may be threaded into the joint 70, by the tool 11.

In this case, the flats 21a of the barbed portion 14a tend to perform a certain amount of cutting of threads, acting somewhat like a self-tapping screw. Thus, the interrupted thread barbs 24a advance like a screw into the joint 70. Upon sufficient penetration, the tool 11 is removed.

Next, the buttress tool 50 is applied to the neck 18 of the anchor 10. Thus, the anchor 10 may be fixed against further penetration into the joint 70. The anchor 10 may be aligned for the proper fit of the barbed portion 14b into the other joint 74 to be joined to the initial joint 70.

One should note that the portions 14a, 14b may be sized in length and diameter according to which joint, the proximal or the intermediate, will first be penetrated. In certain embodiments, either of the anchors 10 illustrated in FIGS. 19 through 25 may be designed to have an angle, or offset such that the axes of one portion 12a, 14a, is not coaxial or collinear with the opposite portion 14, 14b. Necessarily, any threading action must take place before barbs are employed in a linear translation.

Once the penetration of the interrupted-thread barbs 24a is complete, the buttress tool 50 may be put in place on the neck 18, to support force applied linearly (axially) against the barbed portion 14b as the joint 74 is forced onto the barbed portion 14b. Once the face 72 of the joint 74 contacts the face 61 of the buttress tool 50, the buttress tool 50 is removed. The final manual reduction may be completed by placing the faces 68, 72 into contact, subject to the compressive forces imposed by the anchor 10 being in tension.

Referring to FIGS. 26 through 32, while continuing to refer to FIGS. 19 through 25, a procedure for implementing an anchor 10 in accordance with the invention may consider the joint 70 as the proximal phalangeal joint 70 of a toe. Likewise, the joint 74 may be considered the intermediate phalangeal joint 74. The procedure may involve setting the anchor 10 or implant 10 first into the proximal joint 70, and secondly into the intermediate joint 74. Nevertheless, the order may be reversed. That is, the anchor 10 may first be set in the intermediate joint 74, and then in the proximal joint 70. Nevertheless, the description herein simply refers to the joints in the order specified for simplicity.

Following an incision and site preparation, the head or distal end of the proximal joint 70 is removed. Typically, a sagittal saw or other bone cutting tool may be used to remove a portion of the joint 70. Typically, the cut is perpendicular to the axis or the longitudinal axis of the proximal joint 70.

Next, the articular cartilage at the base or the proximal end of the intermediate joint 74 is removed. Again, a sagittal saw or other bone cutting tool may be used. Any remaining areas of bone that are rough or that do not make a smooth and continuous interface may be removed with a small ronguer. The two joints 70, 74 of the toe are then checked for proper reduction and alignment.

For example, the faces 68, 72 should abut smoothly, cleanly, and completely. Likewise, the intermediate joint 74 should align to proceed in the proper direction based on the fitting together of the two faces 68, 72 on the respective joints 70, 74. If necessary, additional cutting may be required or additional tendon work in order to assure a proper reduction and alignment.

An awl may be used to create a pilot hole for the drill in each of the faces 68, 72 of the respective joints 70, 74. The pilot hole 76 may be drilled to make a larger pilot 76, or canal 78 to receive an implant 10 or anchor 10 of the appropriate size. Typically, implant sizing is determined by preoperative templates. Using x-rays, one may determine the appropriate sizing for the anchor 10.

Using the pilot hole 76, a drill forms a receiving aperture 78 sized to receive the implant 10. Typically, the proximal joint 70 is drilled first, followed by the intermediate joint 74. In some circumstances, if the bone is comparatively softer, drilling may not be required for the intermediate joint 74.

Once the receiving aperture 78 or canal 78 has been sized and drilled, the anchor 10 may be implanted by placing it in the correct tool 11. In certain embodiments of a process in accordance with the invention, the tool 11 may be secured to the non penetrating end of the anchor 10. The buttress tool 50 with its keyed aperture 59 or slot 59 at the operational end 56 thereof may be secured to the neck 18 of the anchor 10. In this way, force may be applied to the anchor 10, without the anchor 10 retreating, in an axial direction along the length thereof by the tool 11. The keyed tab or face 61 of the buttress tool 50 prevents over insertion beyond the desired position for the anchor 10. If the anchor 10 is angled between the two portions 14a, 14b or the screw portion 12 and the barbed portion 14, then one must ensure that the unpenetrated end 14b is oriented in the proper direction, pointing plantar and at an appropriate angle to receive the intermediate joint 74.

The anchor 10 or implant 10 should be inserted into the proximal phalanx 70 first in this procedure. If the anchor 10 is difficult to insert, then the channel 78 or canal 78 may be redrilled at a larger diameter using the next larger size of proximal drill. In alternative embodiments, the tool 11 may be used to rotate the anchor 10, and thread it into the canal 76 using the interrupted threads 24a or interrupted-thread barbs 24a.

Nevertheless, in one currently contemplated embodiment, an anchor 10 such as that illustrated in FIGS. 19 through 22 may be threaded into the proximal phalanx 70. Thereafter, the barbs 14 may be penetrated by linear translation into the intermediate phalanx 74 or the intermediate joint 74.

The buttress tool 50 may be used to stabilize the anchor 10 implanted in the proximal joint 70. Then the joint 74 may be distracted against the tension of tendons and placed over the barbs 14, and particularly the barbs 14b, for insertion of the anchor 10 into the intermediate joint 74. Against the supporting force of the face 61, the intermediate joint 74 may be driven over the barbs 14b without risk of the anchor 10 penetrating further into the proximal joint 70.

Once the face 72 of the intermediate joint 74 contacts the face 61 of the tool 50, the tool 50 may be removed. The reduction may be completed manually, placing the two faces 68, 72 into intimate contact, with the anchor 10 maintaining compression therebetween supported by the tension in the anchor 10 itself. Once the joint (both phalanges 70, 74) has been reduced, and contact is secure, the incision may be closed up with a standard closure.

In embodiments where the anchor 10 does not have an angle between its opposing ends 12, 14 or 14a, 14b, and a cannula 27 is provided therein, a K-wire 67 may be used in the surgical technique. In this embodiment of a process in accordance with the invention, the incision is made, the site is prepared, and the head of the proximal phalanx 70 is removed with a sagittal saw or other bone cutting tool. Again, the cut should typically be perpendicular to the longitudinal axis of that proximal joint 70. Typically about four millimeters of bone is removed between the proximal joint 70 and the intermediate joint 74.

The articular cartilage on the base or the proximal end of the intermediate joint 74 (intermediate phalanx 74) is removed with a sagittal saw or bone cutting tool. Rough areas are then removed if required by a small ronguer. Proper alignment and reduction are then checked between the two joints 70, 74 at their respective faces 68, 72. If necessary, tendon work may be required and additional smoothing or fitting.

The implant 10 or anchor 10 should have been sized in advance by reference to a preoperative template, in view of the x-rays taken of the foot. An awl creates a pilot hole 76 for a drill, which is typically about 0.8 millimeters smaller than the outer diameter of the actual implant 10 or anchor 10. In certain embodiments, the outer diameters 32, 34 of the respective ends 12, 14 of the anchor 10 may have different values. Accordingly, the drill should be selected to be appropriately smaller than the outer diameter 32, 34 of an anchor to be applied to a particular joint 70, 74.

A color coordination between the K-wire 67 and the anchor 10 permits selection of the proper K-wire 67. The K-wire 67 may then be integrated or driven through the intermediate phalanx 74 as well as the distal phalanx and through the end of the toe. Again, if a patient has comparatively softer bone, then drilling may not be required for the intermediate phalanx 74. Otherwise, a cannulated drill bit may be run over the top of the K-wire 67 in order to drill down into the intermedullary canal of the intermediate phalanx 74.

The implant 10 may be placed over the K-wire 67 and the keyed tool 50 may be used to apply linear force to insert the implant 10 into the intermediate phalanx 74. Again, the key 59 or slot 59 may actually be used like a wrench in order to thread the interrupted thread barbs 24a, or threads 22 of an anchor 10 into the intermediate phalanx 74. Similarly, a cannulated tool 11 may be used to thread or drive by the barbs 24 or threads 22 into by their joint 70, 74. So long as it is the first to be penetrated.

In this example, the tool 50 may be used to provide a linear axial force along the anchor 10 in order to insert it into the intermediate phalanx 74. The K-wire must then be integrated, drawn down through the anchor 10 until its proximal tip is even with or below the proximal tip of the implant 10. With the K-wire 67 thus out of the way to interfere, the implant 10 may be inserted into the proximal phalanx. The keyed buttress tool 50 secured about the neck 18 of the anchor 10 assures that the anchor 10 may not retreat into the intermediate phalanx 74 further during this reduction.

In the event that the anchor 10 or implant 10 appears difficult to insert proximally, the implant 10 may be removed from the degree of penetration into the proximal phalanx 70, and the canal 78 may be redrilled using the next largest drill bit about the K-wire 67. Thereupon, the anchor 10 or implant 10 may be reinserted into the proximal joint 70, or the canal 78 formed therein. Once the joint has been reduced such that the faces 68, 72 are in contact with the buttress tool 50, the reduction may be completed manually. The toe may be plantarflexed to the desired position, and the K-wire 67 may be retrograded until it is adequately across the medial proximal joint 70. Herein, the word joint has typically referred to the actual bone segments 70, 74, although it is also common to refer to the interface therebetween as the joint.

Likewise, as used herein, reference numerals having trailing alphabetical characters are used to designate specific instances of a particular item that is designated by the reference numeral. That is, the reference numeral may be used to refer to any or all of a particular item. For clarity, a trailing reference letter may follow a reference numeral in order to identify a specific instance of that item. An instance of an item designated generally by the reference numeral, may have a trailing letter in order to distinguish it from other instances of that item. Similarly, figures may be referred to as figure numbers, generally, whereas the reference numeral following a figure number refers to a particular figure, which may contain a different view of the same item.

That is, so long as the outermost layer of the joint 70 remains intact, it will not tend to heal with another bone. However, by resecting the face 68 slightly toward the bone joint 70 (proximal phalangeal joint 70), the face 68 will then be in a condition to heal against another joint 74 (intermediate phalangeal joint 74) of bone. A face 72 of the second bone joint 74 is likewise resected in order to trim it and prepare it to heal against the first joint 70.

Embodiments of an anchor 10 wherein both ends are barbed portions 14a, 14b require linear insertion of the anchor 10 with the tool 11, rather than rotation as required by threading. By a longitudinal push of the first set 14a of barbs 24 into the pilot hole 76, preferably after broaching, force is applied through the tool 11.

The tool 11 is used to drive the point 26 thereof into the first bone joint 70. The neck 18 in double-end-barb configurations is best formed with a slot portion 16 as a shank 16. Slots 16 are recessed into the diameter of the neck 18. The slot 16 is to be engaged by the receiving end 55 of the buttress tool 50.

Accordingly, the barbs 24 engage the medullar portion of the first bone joint 70, until buried flush with the surface 68 or face 68 of the joint 70. The tool 11 may then be removed.

Disengaging the tool 11 from the anchor 10, a surgeon may now place the buttress tool 50 at the neck 18 of the anchor 10. A surgeon relies on the face 61 of the receiving end 55 to buttress the anchor 10 against further insertion into the joint 70 while the second inserted portion 14b is being inserted into the other joint 74.

The cantilevered prongs 40 will move toward one another, thus providing relief on the edges 48 of the barbs 24. However, upon any tendency to come to rest, or to be withdrawn, each of the edges 48 immediately cuts and drives outward due to the undercut shaping thereof. Thus, the barbs 24 provide a positive force, in tension, holding the face 72 of the second joint 74 against the face 68 of the first joint 70 in compression.

After reduction, stability against relative rotation between the joints 70, 74 during healing is provided by the flats 21 of the two barbed portions 14a, 14b. Stability and tensioning in a longitudinal direction (and thus compression to urge union) is provided by the barbs 24. Rotational adjustments may be made by rotating the joint, but will be resisted by the barb portions 14. A cannulated anchor 10 may be used with a K-wire 67, which remains after completion of the surgery and is typically removed only after substantial healing. A K-wire 67 cannot effectively guide or slide along a cannula 27 of an anchor 10 that has a bend or an angle at the neck 18.

Typically, when one is operating without a K-wire as a guide 67, the proximal side of the anchor 10 is inserted first, or the proximal joint 70 receives the anchor 10 first. Then the buttress instrument 50 is used to stabilize the implant and maintain its axial position within the first joint.

Otherwise, the intermediate phalangeal joint 74 may first be penetrated by the anchor 10 after which the K-wire 67 is driven out through the end of the toe but remains within the cannula 27 of the anchor 10. It remains while the reduction is made. It is shortened, by displacement out the intermediate joint 74. Then, by driving the intermediate phalangeal joint 74 with the anchor 10 secured therein and stabilized by the buttressing tool 50, a surgeon inserts the remaining end 14b of the anchor 10 into the opening 78 of the remaining proximal joint 70.

The joints 70, 74 are reduced, with the anchor 10 providing tension. This draws the joints 70, 74 together, each in compression against the other. A standard closure may be applied to the operation area. Suitable dressings and protection may then be provided to protect the K-wire 67 from being dislocated or causing disruption. Thereafter, the K-wire 67 may be removed at the proper stage of healing.

The present invention may be embodied in other specific forms without departing from its principles of operation essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A method comprising:
providing an anchor defining axial, radial, and circumferential directions, the anchor comprising
a first portion comprising a plurality of at least one of threads and first barbs, distributed axially along the anchor, each extending radially to an edge at an outer diameter shared by the plurality, the edges progressing at an angle along a circumferential direction,
a second portion comprising second prongs extending axially and provided with second barbs distributed along the prongs and extending radially,
a neck between and interconnecting the first and second portions,
the first portion, second portion, and neck being formed as a monolithic, homogeneous unit;
accessing the head of a proximal phalangial joint of a subject;
accessing the base of an intermediate phalangial joint of the subject;
penetrating the first portion into one of the head and the base by the rotating the anchor in a forward circumferential direction while applying a linear force in the axial direction;
penetrating the second portion into the other of the head and base by urging them together in linear translation; and
placing the head and base into mutual contact by the urging.

2. The method of claim 1, further comprising:
forming a first interface surface by resecting the head;
forming a second interface surface by resecting the base; and
removing the first portion by rotating in a direction opposite the forward circumferential direction.

3. The method of claim 1, further comprising:
drilling a first pilot in the head through a first interface surface;
drilling a second hole in the base through a second interface surface; and
engaging, by at least two axially adjacent second barbs, a region of cortical bone.

4. The method of claim 1, wherein:
the rotating penetrates the first portion into the head to a depth proximate the neck; and
the linear translation penetrates the second portion into the base.

5. The method of claim 1, wherein:
the first portion comprises first prongs having the first barbs distributed therealong; and
the method further comprises engaging by a cortical portion of one of the first and second joints at least two axially adjacent barbs distributed along the same one of the first and second portions.

6. The method of claim 5, wherein:
the rotating penetrates the first portion into the head to a first depth proximate the neck;
the linear translation penetrates the second portion into the base to a second depth; and
the method further comprises securing the first portion at the first depth by buttressing the neck against linear motion of the first portion with respect to the head.

7. The method of claim 6, further comprising:
deflecting the first barbs radially by bending of the first prongs in response to the engaging by the cortical portion.

8. The method of claim 7, further comprising:
removing the first portion by rotating the first portion in a direction opposite the forward circumferential direction.

9. The method of claim 8, further comprising:
inserting a K-wire into one of the base and the head; and
guiding the anchor by the K-Wire during penetration of at least one of the base and the head.

10. A method of joinder and fixation of independent segments of bone with respect to one another, the method comprising:
providing an arthrodesis anchor defining a axial, radial, and circumferential directions, the anchor comprising
a first portion comprising a plurality of at least one of threads and first barbs, distributed axially along the anchor, each extending radially to an edge at an outer diameter shared by the plurality, the edges progressing at an angle along a circumferential direction,
a second portion comprising second prongs extending axially and provided with second barbs distributed along the prongs and extending radially,
a neck portion connecting the first and second portions,
the first portion, second portion, and neck further formed as a homogeneous monolith;
resecting first and second segments of bone to create respective first and second surfaces;
testing the first segment for resistance to penetration;
selecting, based on the resistance, a selected motion from between a first motion consisting of axial translation and a second motion comprising circumferential rotation;
passing at least some of the first portion through the first surface by the selected motion.

11. The method of claim 10, further comprising:
inserting the second portion through the second surface by axial translation.

12. The method of claim 10, further comprising
urging the first and second surfaces into compression against one another; and
maintaining the compression, by tension in the anchor, between the first and second pluralities of barbs.

13. The method of claim 10, further comprising:
engaging, by at least two axially adjacent first barbs a cortical portion of first segment.

14. The method of claim 10, further comprising:
engaging, by at least two axially adjacent second barbs a cortical portion of second segment.

15. The method of claim 10, further comprising:
placing the second portion into a tool; and
inserting the anchor by applying, with the tool, a force and selected motion to the anchor.

16. The method of claim 10, further comprising:
locking the anchor in place by a buttress tool to resist retreat of the first portion into the first bone segment during insertion of the second portion into the second bone segment.

17. The method of claim 10 wherein the tool is shaped to receive one of the first and second portions such that the other thereof extends co-linearly with the handle end.

18. An arthrodesis anchor system defining a axial, radial, and circumferential directions, the system comprising:
a first portion comprising a plurality of at least one of threads and first barbs, distributed axially along the anchor, each extending radially to an edge at an outer diameter shared by the plurality, the edges progressing at an angle along a circumferential direction,
a second portion comprising second prongs extending axially and provided with second barbs distributed along the prongs and extending radially,
a neck portion connecting the first and second portions,
the first portion, second portion, and neck further formed as a homogeneous monolith.

19. The system of claim 18, further comprising a buttress tool fitted to the neck portion and sized to resist penetration of the first portion beyond the neck.

20. The system of claim 18, further comprising the first, second, and neck portions further provided with a cannula passing therethrough sized to receive a K-wire as a guide in the axial direction.
